(19) United States
(12) Patent Application Publication
(10) Pub. No.: US 2021/0289826 A1
(43) Pub. Date: Sep. 23, 2021

(54) VITAMIN D2-CONTAINING MUSHROOM OIL, AND PREPARATION AND USE THEREOF

(71) Applicant: Hanpeng QU, Changsha (CN)

(72) Inventors: Yun LIANG, Changsha (CN); Sheng CAO, Changsha (CN); Shenjian WANG, Changsha (CN)

(21) Appl. No.: 17/342,965

(22) Filed: Jun. 9, 2021

Related U.S. Application Data
(63) Continuation of application No. PCT/CN2019/101029, filed on Aug. 16, 2019.

(30) Foreign Application Priority Data
Dec. 10, 2018 (CN) 201811505356.5

(51) Int. Cl. A23L 33/155; A23L 5/30; A23D 9/007; A23L 29/00; A23L 31/00; A23D 9/02

(52) U.S. Cl. CPC A23L 33/155; A23L 5/36; A23D 9/02; A23L 29/035; A23L 31/00; A23D 9/007

(57) ABSTRACT

A method for preparing a vitamin D₂-containing mushroom oil. A mushroom sliced to obtain mushroom slices. The mushroom slices are irradiated with an ultraviolet light. The irradiated mushroom slices are subjected to extraction with an edible oil followed by solid-liquid separation to obtain the vitamin D₂-containing mushroom oil. The ultraviolet light is a combination of an ultraviolet B (UVB) light with a wavelength of 280-315 nm and an ultraviolet C (UVC) light with a wavelength of 200-280 nm.

### CROSS-REFERENCE TO RELATED APPLICATIONS

This application is a continuation of International Patent Application No. PCT/CN2019/101029, filed on Aug. 16, 2019, which claims the benefit of priority from Chinese Patent Application No. 201811505356.5, filed on Dec. 10, 2018. The content of the aforementioned applications, including any intervening amendments thereto, is incorporated herein by reference in its entirety.

### TECHNICAL FIELD

This application relates to food processing, and more particularly to a vitamin D₂-containing mushroom oil, and a preparation and use thereof.

### BACKGROUND

Vitamin D is a fat-soluble steroid derivative, and is necessary for human body. Apart from regulating calcium and phosphorus metabolism as a nutritional substance, the vitamin D can also be used in antitumor, prevention of cardiovascular diseases, autoimmune diseases and diabetes. Vitamin D₂ and vitamin D₃ are more closely related to human health. The vitamin D₃ can be obtained by isomerization of 7-dehydrocholesterol in human epidermal cells through exposing to sunlight, whereas the vitamin D₂ cannot be synthesized by the human body. The deficiency of vitamin D₂ may cause crickets and senile osteoporosis. A variety of dietary ways are needed to supply the vitamin D₂ to human body.

Food and sunshine are the main source of vitamin D₂ for human body. However, the content of vitamin D₂ is usually low in the natural food. Lean meat, milk, and nuts contain trace amounts of vitamin D₂, while vegetables, grains and their products and fruits contain little or almost no vitamin D₂. In addition, people with skin diseases or work in the night may not be able to supplement vitamin D₂ through sunbathing.

Vitamin D₂ derived from edible fungi has been widely used in health foods and functional foods. Most of the vitamin D₂ derived from edible fungi are in the form of powders. When the powers are applied into solid products, the mixing uniformity may lead to the content deviation of the target product during the factory inspection. When the powders are applied into liquid products, because the edible fungus is insoluble in water or oil, the powders can only exist in a suspended state in the target product. The problems such as precipitation are prone to occur, which further restricts the promotion of the application. In addition, the edible fungi need to be dried during the processing, which may cause serious degradation or isomerization of vitamin D₂ due to the influence of high temperature and oxygen, and reduce the yield of vitamin D₂.

### SUMMARY

An objective of the present disclosure is to provide a method for preparing a vitamin D₂-containing mushroom oil, so as to overcome the problems in the prior art that the edible mushroom powder is not conducive to product processing, and has deviation in detecting the content of vitamin D₂. The method provided herein can improve the vitamin D₂ content in the mushroom. In addition, since the vitamin D₂ is soluble in the oil, the method extracts the vitamin D₂ from the mushroom into an edible oil, so as to obtain a liquid vitamin D₂, avoiding the detection deviation of vitamin D₂ content in solid food adding with the mushroom. The mushroom oil is convenient to apply in functional food and health food.

To achieve the above-mentioned objectives, in a first aspect, the present disclosure provides a method for preparing a vitamin D₂-containing mushroom oil, which comprises:

slicing a mushroom to obtain mushroom slices; irradiating the mushroom slices with an ultraviolet light; and

subjecting the irradiated mushroom slices to extraction with an edible oil, and followed by solid-liquid separation to obtain the vitamin D₂-containing mushroom oil;

wherein the ultraviolet light is a combination of an ultraviolet B (UVB) light with a wavelength of 280-315 nm and an ultraviolet C (UVC) light with a wavelength of 200-280 nm.

In some embodiments, irradiation with the UVB light is performed at a radiation dose of 1.5-6.5 J/m² for 8-150 min; and irradiation with the UVC light is performed at a radiation dose of 80-120 mJ/m² for 20-30 min.

In some embodiments, the mushroom slices have a thickness of 0.8-1.5 mm.

In some embodiments, the method further comprises:

before the extraction with the edible oil, crushing the irradiated mushroom slices into 80-150 mesh particles.

In some embodiments, the extraction comprises:

adding the edible oil and the irradiated mushroom slices into an extraction tank; vacuumizing the extraction tank; and introducing nitrogen with a purity of no less than 99.9% into the extraction tank to keep a pressure in the extraction tank at 0.02-0.1 MPa.

In some embodiments, the extraction is performed at 10-85° C. for 8-120 h, and a weight ratio of the edible oil to a dry weight of the irradiated mushroom slices is 0.5-30:1.

In some embodiments, the edible oil is selected from the group consisting of a sunflower oil, a rapeseed oil, an olive oil, a corn oil, a camellia seed oil, a soybean oil, a peanut oil and a combination thereof.

In some embodiments, the method further comprises:

before the slicing, washing the mushroom to remove residual culture medium.

In a second aspect, the present disclosure provides a vitamin D₂-containing mushroom oil prepared by the above-mentioned method. The vitamin D₂-containing mushroom oil has a vitamin D₂ content equal to or more than 10 μg/g, a peroxide value no more than 15 meq/kg and an acid value no more than 3 mg/g.

In a third aspect, the present disclosure provides a food comprising the vitamin D₂-containing mushroom oil prepared by the above-mentioned method in food, and the food is a health food or a functional food.

The beneficial effects of the technical solutions are described as follows.

The irradiation using UVB and UVC to the mushroom can effectively promote the conversion of ergosterol in the mushroom into vitamin D₂ and increase the content of vitamin D₂. Meanwhile, the irradiation also sterilizes the mushroom, such that the microbial indicators in the product can meet the requirements of food or pharmaceutical processing. In addition, the vitamin D₂ is soluble in the oil, and thus the vitamin D₂ can be directly extracted from the irradiated mushroom using an edible oil. The mushroom can be crushed into powders before the extraction, so as to facilitate the extraction of vitamin D₂ from the edible oil. Nitrogen is induced in the extraction to isolate oxygen, and the extraction temperature is controlled, so as to prevent the vitamin D₂ from isomerization or degradation. The mushroom oil prepared herein has a content of vitamin D₂ no less than 10 μg/g, a peroxide value no more than 15 meq/kg and an acid value no more than 3 mg/g. The maximum vitamin D₂ content reaches 2154.1 μg/g. The liquid vitamin D₂ is more suitable to apply in food and avoids the deviation of vitamin D₂ content in the product.

### DETAILED DESCRIPTION OF EMBODIMENTS

It should be noted that endpoints and values within ranges disclosed herein are only exemplary, and are intended to include any values close to these values. Any possible combination of numerical values within the range to form one or more new ranges should be considered to be expressly disclosed in this disclosure.

In a first aspect, the present disclosure provides a method for preparing a vitamin D₂-containing mushroom oil, which includes slicing a mushroom to obtain mushroom slices; irradiating the mushroom slices with an ultraviolet light, subjecting the irradiated mushroom slices to extraction with an edible oil followed by solid-liquid separation to obtain the vitamin D₂-containing mushroom oil. The ultraviolet light is a combination of an ultraviolet B (UVB) light with a wavelength of 280-315 nm and an ultraviolet C (UVC) light with a wavelength of 200-280 nm.

The mushroom provided herein is selected from the group consisting of Agaricus bisporus, Lentinus edodes and a combination thereof. The mushroom needs to be refrigerated within 2 days after being harvest. The ultraviolet light is provided by a UVB lamp of 50-100 W and a UVC lamp of 15-40 W. In the ultraviolet light irradiation, the sliced mushroom is placed on a metal mesh tray, and the ultraviolet lamps are fixed a shelf arranged on both side of the tray and 70-80 cm away from the tray, such that the distance between the tray and the ultraviolet light is 20-60 cm, so as to perform a double-sided irradiation. The separation of the edible oil from the extracted mushroom can be a pressure filtration, a suction filtration or any other solid-liquid separation method.

In some embodiments, irradiation with the UVB light is performed at a radiation dose of 1.5-6.5 J/m² for 8-150 min. The radiation dose of the UVB light can be 1.5 J/cm², 2 J/cm², 2.5 J/cm², 3 J/cm², 3.5 J/cm², 4 J/cm², 4.5 J/cm², 5 J/cm², 5.5 J/cm², 6 J/cm², 6.5 J/cm² and any value in a range formed by any two of these point values. Irradiation with the UVC light is performed at a radiation dose of 80-120 mJ/m² for 20-30 min.

In some embodiments, the mushroom slices have a thickness of 0.8-1.5 mm.

In some embodiments, the method further includes

before the extraction with the edible oil, crushing the irradiated mushroom slices into 80-150 mesh particles. In some embodiments, the irradiated mushroom slices is crushed by an ultrafine pulverizer.

In some embodiments, the extraction includes adding the edible oil and the irradiated mushroom slices into an extraction tank, vacuumizing the extraction tank and introducing nitrogen with a purity of no less than 99.9% into the extraction tank to keep a pressure in the extraction tank at 0.02-0.1 MPa.

In some embodiments, the extraction is performed at 10-85° C. for 8-120 h, and a weight ratio of the edible oil to a dry weight of the irradiated mushroom slices is 0.5-30:1. Preferably, the extraction is performed at 10-35° C. The mushroom used herein is fresh mushroom, and a moisture content of the fresh mushroom is about 90%. The dry weight of the mushroom is generally calculated as 10% of the weight of the fresh mushroom.

In some embodiments, the edible oil is selected from the group consisting of a sunflower oil, a rapeseed oil, an olive oil, a corn oil, a camellia seed oil, a soybean oil, a peanut oil and a combination thereof.

In some embodiments, the mushroom is subjected to a pretreatment that a residual culture medium such as straw is removed, and then the mushroom is cleaned through manually cleaning or mechanical cleaning.

In a second aspect, the present disclosure provides a vitamin D₂-containing mushroom oil prepared by the above-mentioned method. The vitamin D₂-containing mushroom oil has a vitamin D₂ content equal to or more than 10 μg/g, a peroxide value no more than 15 meq/kg and an acid value no more than 3 mg/g.

In a third aspect, the present disclosure provides a food comprising the vitamin D₂-containing mushroom oil prepared by the above-mentioned method, and the food is a health food or a functional food.

The present disclosure will be further described below with reference to the accompanying drawings.

In the following examples, the content of vitamin D₂ in the vitamin D₂-containing mushroom oil is measured according to GB 5009.82 using a high performance liquid chromatograph of L-7000 produced by Hitachi, Ltd. (Japan). The recovery rate of extracted vitamin D₂=(the content of vitamin D₂ in the mushroom oil×the quality of mushroom oil)/(the dry weight of mushroom×the content of vitamin D₂ in the dry mushroom)×100%. The peroxide value is measured according to GB5009.227-2016. The acid value is measured according to GB5009 0.229-2016.

Agaricus bisporus and Lentinus edodes are purchased from Shandong Deze Agricultural Technology Co., Ltd. (China). The sunflower oil, rapeseed oil, olive oil, corn oil, camellia seed oil, soybean oil and peanut oil are purchased from COFCO (China).

### Example 1

(1) Slicing

Freshly harvested Agaricus bisporus was cleaned to remove the residual culture medium. 20 kg of the Agaricus bisporus was cut into slices with a thickness of 0.8 mm.

(2) Ultraviolet Light Irradiation

Double sides of the Agaricus bisporus slices were irradiated with ultraviolet B (UVB) light with a wavelength of 280 nm at an irradiation dose of 1.5 J/cm² for 180 min, and then irradiated with ultraviolet C (UVC) light with a wavelength of 200 nm at an irradiation dose of 80 mJ/cm² for 30 min.

(3) Crushing

The irradiated Agaricus bisporus slices were crushed into 80-150 mesh particles.

(4) Extraction

The crushed Agaricus bisporus (dry weight: 2 kg) and 1 kg of a sunflower oil were mixed in an extraction tank. After the extraction tank was vacuumized, nitrogen with a purity of no less than 99.9% was introduced to keep a pressure in the extraction tank at 0.06 MPa. The extraction was performed at 25° C. for 120 h.

(5) Solid-Liquid Separation

The mixture of the sunflower oil and the Agaricus bisporus material was subjected to solid-liquid separation using a plate and frame filter press to obtain a vitamin D₂-containing mushroom oil.

### Example 2

(1) Slicing

Freshly harvested Agaricus bisporus was cleaned to remove the residual culture medium. 20 kg of the Agaricus bisporus was cut into slices with a thickness of 1.2 mm.

(2) Ultraviolet Light Irradiation

Double sides of the Agaricus bisporus slices were irradiated with ultraviolet B (UVB) light with a wavelength of 300 nm at an irradiation dose of 4 J/cm² for 100 min, and then irradiated with ultraviolet C (UVC) light with a wavelength of 240 nm at an irradiation dose of 100 mJ/cm² for 25 min.

(3) Crushing

The irradiated Agaricus bisporus slices were crushed into 80-150 mesh particles.

(4) Extraction

The crushed Agaricus bisporus (dry weight: 2 kg) and 30 kg of a rapeseed oil were mixed in an extraction tank. After the extraction tank was vacuumized, nitrogen with a purity of no less than 99.9% was introduced to keep a pressure in the extraction tank at 0.02 MPa. The extraction was performed at 10° C. for 75 h.

(5) Solid-Liquid Separation

The mixture of the rapeseed oil and the Agaricus bisporus material was subjected to solid-liquid separation using a plate and frame filter press to obtain a vitamin D₂-containing mushroom oil.

### Example 3

(1) Slicing

Freshly harvested Lentinus edodes was cleaned to remove the residual culture medium. 20 kg of the Lentinus edodes was cut into slices with a thickness of 1.5 mm.

(2) Ultraviolet Light Irradiation

Double sides of the Lentinus edodes slices were irradiated with ultraviolet B (UVB) light with a wavelength of 315 nm at an irradiation dose of 6.5 J/cm² for 8 min, and then irradiated with ultraviolet C (UVC) light with a wavelength of 280 nm at an irradiation dose of 120 mJ/cm² for 20 min.

(3) Crushing

The irradiated Lentinus edodes slices were crushed into 80-150 mesh particles.

(4) Extraction

The crushed Lentinus edodes (dry weight: 2 kg) and 60 kg of a corn oil were mixed in an extraction tank. After the extraction tank was vacuumized, nitrogen with a purity of no less than 99.9% was introduced to keep a pressure in the extraction tank at 0.1 MPa. The extraction was performed at 35° C. for 8 h.

(5) Solid-Liquid Separation

The mixture of the corn oil and the Lentinus edodes material was subjected to solid-liquid separation using a suction filtration machine to obtain a vitamin D₂-containing mushroom oil.

### Example 4

(1) Slicing

Freshly harvested Agaricus bisporus was cleaned to remove the residual culture medium. 20 kg of the Agaricus bisporus was cut into slices with a thickness of 1 mm.

(2) Ultraviolet Light Irradiation

Double sides of the Agaricus bisporus slices were irradiated with ultraviolet B (UVB) light with a wavelength of 305 nm at an irradiation dose of 8 J/cm² for 10 min, and then irradiated with ultraviolet C (UVC) light with a wavelength of 200 nm at an irradiation dose of 150 mJ/cm² for 15 min.

(3) Crushing

The irradiated Agaricus bisporus slices were crushed into 80-150 mesh particles.

(4) Extraction

The crushed Agaricus bisporus (dry weight: 2 kg) and 15 kg of a soybean oil were mixed in an extraction tank. After the extraction tank was vacuumized, nitrogen with a purity of no less than 99.9% was introduced to keep a pressure in the extraction tank at 0.1 MPa. The extraction was performed at 65° C. for 100 h.

(5) Solid-Liquid Separation

The mixture of the soybean oil and the Agaricus bisporus material was subjected to solid-liquid separation using a plate and frame filter press to obtain a vitamin D₂-containing mushroom oil.

### Example 5

(1) Slicing

Freshly harvested Lentinus edodes was cleaned to remove the residual culture medium. 20 kg of the Lentinus edodes was cut into slices with a thickness of 2 mm.

(2) Ultraviolet Light Irradiation

Double sides of the Lentinus edodes slices were irradiated with ultraviolet B (UVB) light with a wavelength of 290 nm at an irradiation dose of 3 J/cm² for 150 min, and then irradiated with ultraviolet C (UVC) light with a wavelength of 220 nm at an irradiation dose of 90 mJ/cm² for 30 min.

(3) Crushing

The irradiated Lentinus edodes slices were crushed into 80-150 mesh particles.

(4) Extraction

The crushed Lentinus edodes (dry weight: 2 kg) and 0.5 kg of an olive oil were mixed in an extraction tank. After the extraction tank was vacuumized, nitrogen with a purity of no less than 99.9% was introduced to keep a pressure in the extraction tank at 0.05 MPa. The extraction was performed at 85° C. for 8 h.

(5) Solid-Liquid Separation

The mixture of the olive oil and the Lentinus edodes material was subjected to solid-liquid separation using a plate and frame filter press to obtain a vitamin D₂-containing mushroom oil.

### Example 6

(1) Slicing

Freshly harvested Agaricus bisporus and Lentinus edodes was cleaned to remove the residual culture medium. 10 kg of the Agaricus bisporus and 10 kg Lentinus edodes was cut into slices with a thickness of 1.5 mm.

(2) Ultraviolet Light Irradiation

Double sides of the slices were irradiated with ultraviolet B (UVB) light with a wavelength of 285 nm at an irradiation dose of 3.5 J/cm² for 90 min, and then irradiated with ultraviolet C (UVC) light with a wavelength of 205 nm at an irradiation dose of 110 mJ/cm² for 25 min.

(3) Extraction

The irradiated Agaricus bisporus and Lentinus edodes (dry weight: 2 kg), 20 kg of a camellia seed oil and 20 kg of a peanut oil were mixed in an extraction tank. After the extraction tank was vacuumized, nitrogen with a purity of no less than 99.9% was introduced to keep a pressure in the extraction tank at 0.15 MPa. The extraction was performed at 60° C. for 60 h.

(4) Solid-Liquid Separation

The mixture of the camellia seed oil, the peanut oil, the Agaricus bisporus and the Lentinus edodes was subjected to solid-liquid separation using a plate and frame filter press to obtain a vitamin D₂-containing mushroom oil.

### Example 7

(1) Slicing

Freshly harvested Lentinus edodes was cleaned to remove the residual culture medium. 20 kg of the Lentinus edodes was cut into slices with a thickness of 2.5 mm.

(2) Ultraviolet light irradiation

Double sides of the Lentinus edodes slices were irradiated with iltraviolet B (UVB) light with a wavelength of 295 nm at an irradiation dose of 4.5 J/cm² for 90 min, and then irradiated with ultraviolet C (UVC) light with a wavelength of 215 nm at an irradiation dose of 110 mJ/cm² for 25 min.

(3) Extraction

The irradiated Lentinus edodes (dry weight: 2 kg), 15 kg of a sunflower oil, 15 kg of a rapeseed oil and 15 kg of a soybean oil were mixed in an extraction tank. After the extraction tank was vacuumized, nitrogen with a purity of no less than 99.9% was introduced to keep a pressure in the extraction tank at 0.05 MPa. The extraction was performed at 60° C. for 60 h.

(4) Solid-Liquid Separation

The mixture of the sunflower oil, the rapeseed oil, the soybean oil and the Lentinus edodes material was subjected to solid-liquid separation using a plate and frame filter press to obtain a vitamin D₂-containing mushroom oil.

### Comparative Example 1

(1) Slicing

Freshly harvested Agaricus bisporus was cleaned to remove the residual culture medium. 20 kg of the Agaricus bisporus was cut into slices with a thickness of 0.8 mm.

(2) Crushing

The Agaricus bisporus slices were crushed into 80-150 mesh particles.

(3) Extraction

The crushed Agaricus bisporus (dry weight: 2 kg), 15 kg of a sunflower oil and 15 kg of a rapeseed oil were mixed in an extraction tank. After the extraction tank was vacuumized, nitrogen with a purity of no less than 99.9% was introduced to keep a pressure in the extraction tank at 0.06 MPa. The extraction was performed at 25° C. for 100 h.

(4) Solid-Liquid Separation

The mixture of the sunflower oil, the rapeseed oil and the Agaricus bisporus material was subjected to solid-liquid separation using a plate and frame filter press to obtain a vitamin D₂-containing mushroom oil.

### Comparative Example 2

(1) Slicing

Freshly harvested Agaricus bisporus was cleaned to remove the residual culture medium. 20 kg of the Agaricus bisporus was cut into slices with a thickness of 0.8 mm.

(2) Ultraviolet Light Irradiation

Double sides of the Agaricus bisporus slices were irradiated with ultraviolet B (UVB) light with a wavelength of 280 nm at an irradiation dose of 1.5 J/cm² for 100 min, and then irradiated with ultraviolet C (UVC) light with a wavelength of 200 nm at an irradiation dose of 80 mJ/cm² for 30 min.

(4) Drying: The irradiated Agaricus bisporus was dried with hot air at 200° C. for 150 min.

(3) Crushing

The dried Agaricus bisporus slices were crushed into 80-150 mesh particles.

(4) Extraction

The crushed Agaricus bisporus (dry weight: 2 kg) and 6 kg of a soybean oil were mixed in an extraction tank for mixing. The extraction was performed at room temperature and pressure for 100 h.

(5) Solid-Liquid Separation

The mixture of the soybean oil and the Agaricus bisporus material was subjected to solid-liquid separation using a plate and frame filter press to obtain a vitamin D₂-containing mushroom oil.

### Experimental Results

The mushroom oils containing vitamin D₂ prepared in Examples 1-7 and Comparative Examples 1-2 were respectively analyzed for the vitamin D₂ content in the mushroom oil, the recovery rate of the extracted vitamin D₂, the peroxide value and the acid value of the mushroom oil. The measured data were shown in Table 1.

TABLE 1
Vitamin D₂ content in the mushroom oil, recovery rate
of the extracted vitamin D₂, peroxide value and
acid value of the mushroom oil

| | Vitamin D₂ | Recovery | | |
|---|---|---|---|---|
| | content in the | rate of the | | |
| | mushroom | extracted | Peroxide | Acid |
| | oil | vitamin D₂ | value | value |
| Sample | (μg/g) | (%) | (meq/kg) | (mg/g) |
| Example 1 | 2154.1 | 87.1 | 4.76 | 0.76 |
| Example 2 | 40.98 | 96.7 | 4.15 | 0.73 |
| Example 3 | 19.94 | 97.5 | 3.87 | 0.59 |
| Example 4 | 45.88 | 96.3 | 5.17 | 0.81 |
| Example 5 | 1473.17 | 79.7 | 5.35 | 0.84 |
| Example 6 | 19.24 | 89.8 | 11.28 | 2.24 |
| Example 7 | 15.76 | 88.9 | 12.07 | 1.81 |
| Comparative | 1.22 | 96.8 | 6.1 | 2.13 |
| Example 1 | | | | |
| Comparative | 9.68 | 82.3 | 16.1 | 3.49 |
| Example 2 | | | | |

Table 1 shows that in Examples 1-7 using the method of present disclosure, the edible oil can effectively extract vitamin D₂ in the mushroom, and the vitamin D₂ content in mushroom oil is up to 2154.1 μg/g. Comparing Comparative Example 1 with Examples 1-7, it can be seen that the irradiation using UVB and UVC to the mushroom can effectively promote the conversion of ergosterol in the mushroom into vitamin D₂ and increase the content of vitamin D₂. Comparing Comparative Example 2 with Examples 1-7, it can be seen that without high-temperature drying, the vitamin D₂ in the mushroom is directly extracted with the edible oil to isolate oxygen and control the extraction temperature, which can effectively prevent the vitamin D₂ from isomerization or degradation.

The above-mentioned embodiments are only preferred embodiments, and not intended to limit the scope of the disclosure. It should be noted that variations and modifications made by those skilled in the art without departing from the spirit of the disclosure should fall within the scope of the disclosure defined by the appended claims.

What is claimed is:

1. A method for preparing a vitamin D₂-containing mushroom oil, comprising:
slicing a mushroom to obtain mushroom slices; irradiating the mushroom slices with an ultraviolet light; and
subjecting the irradiated mushroom slices to extraction with an edible oil followed by solid-liquid separation to obtain the vitamin D₂-containing mushroom oil;
wherein the ultraviolet light is a combination of an ultraviolet B (UVB) light with a wavelength of 280-315 nm and an ultraviolet C (UVC) light with a wavelength of 200-280 nm.

2. The method of claim 1, wherein irradiation with the UVB light is performed at a radiation dose of 1.5-6.5 J/m² for 8-150 min; and irradiation with the UVC light is performed at a radiation dose of 80-120 mJ/m² for 20-30 min.

3. The method of claim 1, wherein the mushroom slices have a thickness of 0.8-1.5 mm.

4. The method of claim 1, further comprising:
before the extraction with the edible oil, crushing the irradiated mushroom slices into 80-150 mesh particles.

5. The method of claim 1, wherein the extraction comprises:
adding the edible oil and the irradiated mushroom slices into an extraction tank; vacuumizing the extraction tank; and introducing an inert gas into the extraction tank to keep a pressure in the extraction tank at 0.02-0.1 MPa.

6. The method of claim 1, wherein the extraction is performed at 10-85° C. for 8-120 h, and a weight ratio of the edible oil to a dry weight of the irradiated mushroom slices is 0.5-30:1.

7. The method of claim 5, wherein the extraction is performed at 10-85° C. for 8-120 h, and a weight ratio of the edible oil to a dry weight of the irradiated mushroom slices is 0.5-30:1.

8. The method of claim 6, wherein the edible oil is selected from the group consisting of a sunflower oil, a rapeseed oil, an olive oil, a corn oil, a camellia seed oil, a soybean oil, a peanut oil and a combination thereof.

9. The method of claim 7, wherein the edible oil is selected from the group consisting of a sunflower oil, a rapeseed oil, an olive oil, a corn oil, a camellia seed oil, a soybean oil, a peanut oil and a combination thereof.

10. The method of claim 1, further comprising:
before the slicing, washing the mushroom to remove residual culture medium.

11. A vitamin D₂-containing mushroom oil prepared by the method of claim 1, wherein the vitamin D₂-containing mushroom oil has a vitamin D₂ content equal to or more than 10 μg/g, a peroxide value no more than 15 meq/kg and an acid value no more than 3 mg/g.

12. A food comprising the vitamin D₂-containing mushroom oil of claim 11, wherein the food is a health food or a functional food.
